(19) United States
(12) Patent Application Publication
(10) Pub. No.: US 2022/0162113 A1
(43) Pub. Date: May 26, 2022

(54) BORATE-GLASS COMPOSITIONS, METHODS OF MANUFACTURE, AND USES

(71) Applicant: THE ROYAL INSTITUTION FOR THE ADVANCEMENT OF LEARNING/MCGILL UNIVERSITY, Montreal (CA)

(72) Inventors: Showan N. NAZHAT, Montreal (CA); William C. LEPRY, Montreal (CA); Shiva NASERI, Montreal (CA)

(21) Appl. No.: 17/600,804

(22) PCT Filed: Mar. 31, 2020
(86) PCT No.: PCT/CA2020/050421

§ 371 Date: Oct. 1, 2021

Related U.S. Application Data
(60) Provisional application No. 62/827,730, filed on Apr. 1, 2019.

(51) Int. Cl. C03C 3/155

(52) U.S. Cl. CPC C03C 3/155; A61K 47/6903; C03C 2203/20

(57) ABSTRACT

Compositions comprising a sol-gel derived glass, the sol-gel derived glass comprising two main components, the main components comprising a borate component and an alkaline earth metal component. Methods of making the compositions comprising combining precursor solutions containing boron ions, with alkaline earth metal ions to form a solution; gelling the solution to form a gel; drying the gel; and calcining the dried gel.

### FIELD OF THE TECHNOLOGY

The present technology relates generally to borate-glass compositions, methods of manufacture, and uses thereof, specifically but not exclusively, borate-glass compositions for use as biomaterials.

### BACKGROUND OF THE TECHNOLOGY

Biomaterials are used for the repair, replacement, construction or augmentation of hard and soft tissue in response to diseases, and trauma. Biomaterials are also used for cosmetic enhancement, as well as drug delivery vehicles.

Glass compositions used as biomaterials can be any one of silicate-based, phosphate-based and borate-based.

Borate-based glasses have recently gained in popularity due in part to their fast degradation rate and their potential to be fully biodegradable when compared to traditional silicate glasses.

In formulating new borate glass compositions for specific biomedical uses, predictability and control of any induced physiological response is desirable. However, many factors contribute to the induced physiological response of a bioactive glass composition, such as chemical composition, rate of dissolution, surface area, porosity etc.

It is desired to provide improved or alternative borate-based glass compositions for certain biomedical uses, and their methods of manufacture.

### SUMMARY OF THE TECHNOLOGY

Aspects and embodiments of the present disclosure provide borate-glass compositions for use as biomaterials, methods of their manufacture, and uses of the same. The term “borate” as used herein means an oxide of boron including but not limited to B₂O₃.

From a first aspect, there is a provided a composition comprising a sol-gel derived glass, the sol-gel derived glass comprising two main components, the two main components comprising a borate component and an alkaline earth metal component.

In certain embodiments, the alkaline earth metal component is selected from a calcium component, a magnesium component and a calcium-magnesium component. In certain embodiments, the alkaline earth metal component is a calcium oxide component, a magnesium oxide component, and a calcium oxide-magnesium oxide component.

In certain embodiments, the sol-gel derived glass is substantially silica free and substantially phosphate free.

In certain embodiments, the composition further comprises a doping component selected from silver, titanium, lithium, silicon, gold, copper, cobalt, fluoride, iron, manganese, molybdenum, magnesium, nickel, rubidium, strontium, potassium, zinc, niobium, cesium, and gallium.

In certain embodiments, the doping component is less than about 10 weight %, or less than about 5 weight % of the composition.

In certain embodiments, the composition is bioactive.

In certain embodiments, the alkaline earth metal component is calcium oxide and the composition can undergo mineralization.

In certain embodiments, the composition can at least partially convert to hydroxyapatite or calcite, optionally wherein conversion comprises dissolution and precipitation, and optionally wherein a conversion rate ranges from about 30 minutes to about 36 hours, as measured by in vitro testing in simulated body fluid and analyzed by x-ray diffraction.

In certain embodiments, the composition has an antibacterial effect.

In certain embodiments, the alkaline earth metal component comprises calcium oxide, and the calcium oxide is the main component of the glass system based on weight %.

In certain embodiments, the calcium oxide component is equal to or more than about 50 weight %, or is about 50 weight % to about 70 weight %.

In certain embodiments, the borate component is the main network forming component based on weight %.

In certain embodiments, the borate component is equal to or more than about 50 weight %, or is about 50 weight % to about 85 weight %.

In certain embodiments, the composition solubilizes to promote wound healing.

In certain embodiments, the composition further comprises a carrier, wherein the carrier is a paste, liquid or gel.

In certain embodiments, the carrier comprises one or more of glycerine, polyethylene glycol, titanium dioxide, and syloid.

In certain embodiments, the composition further comprises a bioactive agent, optionally wherein the bioactive agent is selected from one or more of cells, genes, drug molecules, therapeutic agents, particles, osteogenic agents, osteoconductive agents, osteoinductive agents, anti-inflammatory agents, antibiotics, anticoagulants, and growth factors.

In certain embodiments, the sol-gel derived glass has a surface area per mass of more than: about 1 m²/g, more than about 1 m²/g, more than about 5 m²/g; more than about 10 m²/g, more than about 20 m²/g, more than about 30 m²/g, more than about 40 m²/g, more than about 50 m²/g; about 5-300 m²/g, 10-300 m²/g, 20-300 m²/g, 30-300 m²/g, 40-300 m²/g, 50-300 m²/g, 60-300 m²/g, 70-300 m²/g, 80-300 m²/g, 90-300 m²/g, 100-300 m²/g, 110-300 m²/g, 120-300 m²/g, 130-300 m²/g, 140-300 m²/g, 150-300 m²/g, 200-300 m²/g, 250-300 m²/g, 5-250 m²/g, 5-200 m²/g, 5-150 m²/g or 5-100 m²/g.

In certain embodiments, the sol-gel derived glass has a pore volume per mass of: more than about 0.001 cm³/g, more than about 0.01 cm³/g, more than about 0.02 cm³/g, more than about 0.03 cm³/g, more than about 0.04 cm³/g, more than about 0.05 cm³/g, more than about 0.06 cm³/g, more than about 0.07 cm³/g, more than about 0.08 cm³/g, more than about 0.09 cm³/g, more than about 0.1 cm³/g, more than about 0.2 cm³/g, more than about 0.3 cm³/g, more than about 0.4 cm³/g; between about 0.1-3.0 cm³/g, 0.2-3.0 cm³/g, 0.3-3.0 cm³/g, 0.4-3.0 cm³/g, 0.5-3.0 cm³/g, 0.6-3.0 cm³/g, 0.7-3.0 cm³/g, 0.8-3.0 cm³/g, 0.9-3.0 cm³/g, 1.0-3.0 cm³/g, 0.1-2.5 cm³/g, 0.42-1.18 cm³/g or 0.1-2.0 cm³/g.

In certain embodiments, the sol-gel derived glass is amorphous, crystalline or semi-crystalline.

In certain embodiments, the composition is in particulate form, and optionally wherein the particles range in diameter from 0.2-1 μm, 5-2000 μm, 5-100 μm, or 25-75 μm.

From a further aspect, there is provided a method for making embodiments of the composition as described herein, the method comprising combining precursor solutions containing boron ions, with alkaline earth metal ions to form a solution; gelling the solution to form a gel; drying the gel; and calcining the dried gel.

In certain embodiments, the alkaline earth metal ions comprise calcium ions, magnesium ions, or calcium and magnesium ions.

In certain embodiments, the precursor solution containing boron ions is selected from trimethyl borate B(OCH₃)₃, triethyl borate B(C₂H₅O)₃, tributyl borate B(CH₃(CH₂)₃O)₃, Tri-tert-butyl borate (B₃(CH₃)₃CO) and boric acid, dissolved methanol or ethanol.

In certain embodiments, wherein the precursor solution containing calcium ions is selected from Calcium nitrate tetrahydrate (Ca(NO₃)₂4H₂O), Calcium Chloride (CaCl₂), Calcium Ethoxide (Ca(C₂H₅O)₂), Calcium methoxide (C₂H₆CaO₂), Calcium methoxyethoxide 5-40% but preferably 20% in methoxyethanol (C₆H₁₄CaO₄), Calcium citrate (Ca₃(C₆H₅O₇)₂), Calcium citrate tetrahydrate (C₁₂H₁₈Ca₃O₁₈), Calcium lactate monohydrate (C₆H₁₂CaO₇), Calcium lactate pentahydrate (C₆H₂₀CaO₁₁), Calcium lactate trihydrate (C₆H₁₆CaO₉), and Calcium lactate gluconate (C₉H₁₆CaO₁₀).

In certain embodiments, the method further comprises grinding the calcined dried gel.

In certain embodiments, gelling the solution comprises maintaining the solution at a temperature between about room temperature and about 60° C., preferably at about 37° C.

In certain embodiments, drying the gel comprises heating the gel and/or allowing loss of humidity to form a dry gel.

In certain embodiments, calcining the dry gel comprises heating the dry gel to between about 400-600° C., or about 100-400° C.

In certain embodiments, the heating comprises using a 3° C/min heating rate, followed by a 2 hour dwell, and then furnace cooling.

In certain embodiments, the method further comprises adjusting the pH of the solution to about 10.5 to about 14.0 or about 11 to about 13.5.

In certain embodiments, the precursor solutions include a doping ion containing solution, and optionally wherein the doping ion is silver.

In certain embodiments, the method further comprises modifying the pH of the solution before adding the precursor solution.

From a further aspect, there is provided a composition comprising a sol-gel derived glass and a doping component, the sol-gel derived glass consisting of a borate component and an alkaline earth metal oxide component, and the doping component comprising silver.

In certain embodiments, the alkaline earth metal oxide component is selected from a calcium oxide component, a magnesium oxide component, and a calcium oxide-magnesium oxide component.

In certain embodiments, the sol-gel derived glass is substantially silica free and substantially phosphate free.

From a yet further aspect, there is provided a method for making the composition as described herein by sol-gel, the method comprising combining a first precursor solution containing boron ions, a second precursor solution containing the alkaline earth metal oxide component of the composition and a third precursor solution containing silver ions to form a solution; gelling the solution to form a gel; drying the gel; and calcining the dried gel, wherein the third precursor solution containing silver ions is added to one or both of the first and second precursor solutions with an acidic pH.

In certain embodiments, the third precursor solution containing silver ions is added to the first precursor solution containing boron ions, and before adding the second precursor solution containing the alkaline earth component.

In certain embodiments, the alkaline earth metal oxide component is calcium oxide or magnesium oxide.

Two-component sol-gel derived glass compositions based on a borate component and an alkaline earth metal component provide a wide range of bioactive properties, which can be controlled and predicted. For example, certain embodiments of a two-component composition based on borate and calcium oxide can, surprisingly, convert to hydroxyapatite and therefore has uses in mineralizing biomedical applications (e.g. for dentin hypersensitivity, craniofacial and bone regeneration). Whereas, in certain other embodiments, a two-component composition based on borate and magnesium oxide is also bioactive but with significantly slower, and in certain cases no hydroxyapatite conversion compared to the borate and calcium oxide composition, and therefore has uses in non-mineralizing biomedical applications (e.g. wound healing). These compositions can be readily modified with doping agents, such as those having antibacterial functionalities, thereby broadening the bioactivity and possible uses. Doping agents with anti-inflammatory and angiogenic properties can also be used.

Surprisingly, for certain embodiments of the composition based on borate and calcium oxide, the inventors found that calcite mineralisation was initiated within 30 minutes (30 mol %B₂O₃-70 mol %CaO; and 40 mol %B₂O₃-60 mol %CaO), and hydroxyapatite mineralization was initiated within 2 hours (for 50 mol %B₂O₃-50 mol %CaO, 60 mol %B₂O₃-40 mol %CaO, and 70 mol % B₂O₃-30 mol %CaO) of contact with simulated body fluid according to x-ray diffraction. Surface mineralisation was observed within 30 minutes of contact with simulated body fluid for all compositions except with the highest borate content) according to ATR-FTIR. To the inventors' knowledge, this was surprising, as two component borate-glass systems have not been shown to mineralize, let alone the ability to control calcite or hydroxyapatite formation based on the composition.

Advantageously, certain embodiments of the present compositions and methods allow for greater flexibility in composition because of the low processing temperature of sol-gel compared to melt-quench methods, and hybrid materials are easily obtained. In embodiments of the present method, the glassy network is created by hydrolysis and condensation reactions of liquid precursors which are typically metal alkoxides. Furthermore, embodiments of the present method allow for tailoring of the surface area, porosity, doping component and hence bioactivity of the resultant biomaterials for a targeted cellular response. Compared to traditional melt-quench equivalent glasses, present sol-gel processing methods can generate glasses with at least 400 and 800 times greater surface area and porosity values in certain embodiments. In certain other embodiments, the surface area and porosity are about 5 to 10 times greater in sol-gel made glasses compared to those made by melt-quenching.

As demonstrated in the Examples, sol-gel derived, borate glasses suitable for biomedical applications such as bone tissue engineering applications and wound healing applications have been created. As demonstrated in the Examples, certain compositions of the present technology provide the ability to rapidly form hydroxyapatite in vitro using simulated body fluid, and cell compatibility was observed demonstrating that these compositions can be used in vivo. Furthermore, similar results are expected with alkali metal fluorides.

### Definitions:

The term “Surface area by mass” as used herein means: The total external surface area (m²) related to the mass (g) of the material. Also referred to as “specific surface area” and expressed as (m²/g).

The term “Pore volume” as used herein means: The total volume of the pores (cm³) within a set amount of material (g). Often expressed as cm³/g.

The term “biomaterial” as used herein means: a material that is biocompatible with a human or animal body when in contact with the body such as by implantation, injection, topical or any other contact. It can be in liquid, gel or solid form.

### BRIEF DESCRIPTION OF THE DRAWINGS

Further aspects and advantages of the present technology will become better understood with reference to the description in association with the following in which:

FIGS. 1A and 1B show, respectively ATR-FTIR spectra and XRD diffractograms of the compositions of Example 1, according to embodiments of the present technology;

FIGS. 2A and 2B show, respectively, NMR stack plot and B4 coordination graphs of the compositions of Example 1, according to embodiments of the present technology;

FIGS. 3A, 3B and 3C show, respectively, pH changes over time, ion release of boron and ion release of calcium, of the compositions of Example 1 when submerged (immersed) in simulated body fluid, according to embodiments of the present technology;

FIG. 4 shows ATR-FTIR spectra of the compositions of Example 1 as a function of immersion time in simulated body fluid, according to embodiments of the present technology;

FIG. 5 shows XRD diffractograms of the compositions of Example 1 as a function of immersion time in simulated body fluid, according to embodiments of the present technology;

FIG. 6 shows scanning electron micrographs of the compositions of Example 1 as a function of immersion time in simulated body fluid, according to embodiments of the present technology;

FIG. 7A and 7B show cell metabolic activity of the compositions of Example 1 at two different concentrations (0.375 mg/mL and 0.75 mg/mL) respectively, according to embodiments of the present technology;

FIGS. 8A and 8B show cell viability of the compositions of Example 1 at two different concentrations (0.375 mg/mL and 0.75 mg/mL) respectively, according to embodiments of the present technology;

FIGS. 9A and 9B show optical density with time of one of the compositions of Example 8 with silver doping, at two different concentrations (1.5 mg/mL and 0.75 mg/mL) respectively, according to embodiments of the present technology;

FIG. 10 shows migration of HaCat cells cultured with the compositions of Example 8, according to embodiments of the present technology;

FIGS. 11A and 11B show, respectively ATR-FTIR spectra and XRD diffractograms of the compositions of Example 8, according to embodiments of the present technology; and

FIG. 12 shows XRD diffractograms of the composition of Example 9 after 1 day immersion in simulated body fluid, according to embodiments of the present technology.

### DETAILED DESCRIPTION OF THE TECHNOLOGY

This technology is not limited in its application to the details of construction and the arrangement of components set forth in the following description or illustrated in the drawings. The technology is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the following description, the same numerical references refer to similar elements. In the drawings, like reference characters designate like or similar parts.

The present technology is directed to sol-gel derived borate-based glass compositions. The compositions are suitable for a range of biomedical uses, including mineralization uses such as for preventing or treating dentine hypersensitivity, regenerating bone, as well as for non-mineralizing applications such as wound healing and cosmetic treatments.

### Compositions

Compositions of the present technology are based on borate-based glass components. In certain embodiments, the borate-based glass compositions have two main components: a borate component and an alkaline earth metal oxide component. The compositions may also include a doping component. The alkaline earth metal oxide component can be calcium, magnesium, strontium, barium or radium.

### Borate-Calcium Glasses

According to certain embodiments of the present technology, glass compositions have two components: a borate component and a calcium oxide component. In certain embodiments of these binary compositions, xB₂O₃.yCaO., x is from about 30-80 mol%, and y is from about 20-70 mol%. In certain other embodiments, x is from about 20-90 mol% and y is from about 10-80 mol%.

Examples of the two component borate glasses including a borate component and a calcium oxide component are provided in Table 1 below:

TABLE 1
Example borate-calcium oxide glass compositions

| | Borate component | Calcium oxide component |
|---|---|---|
| Glass | content/molar % | content/molar % |
| composition | (weight %) | (weight %) |
| B30 | 30 (34.7) | 70 (65.3) |
| B40 | 40 (45.3) | 60 (54.7) |
| B50 | 50 (55.4) | 50 (44.6) |
| B60 | 60 (65.1) | 40 (34.9) |
| B70 | 70 (74.3) | 30 (25.7) |
| B80 | 80 (83.2) | 20 (16.8) |

In certain embodiments, these two component systems including a doping component. The doping component can be trace amounts. Alternatively, the doping component can be more than a trace amount. The doping component can be any one or more of silver, titanium, lithium, silicon, gold, copper, cobalt, fluoride, iron, manganese, molybdenum, magnesium, nickel, rubidium, strontium, potassium, zinc, niobium, cesium, and gallium. Other doping components are possible.

The doping content is less than the borate content and the calcium content in certain embodiments. In certain embodiments of any of the foregoing or following, the doping component comprises about 0.05 to about 10 mol. % or about 0.05 to about 30 mol. % of the composition. The content of silver oxide as the doping agent is determined by potential toxic levels. In certain embodiments, the content of silver oxide as the doping agent is about 0.1-10 mol. %, about 0.1-0.5 mol. %, about 0.5-1 mol. %, or about 1-2 mol. %.

Table 2 shows example borate-calcium oxide compositions including a silver doping component.

TABLE 2
Example borate-calcium oxide compositions including silver as
a doping component

| | | Calcium oxide | |
|---|---|---|---|
| | Borate (B₂O₃) | (CaO) | Silver oxide |
| | component | component | (Ag₂O) doping |
| | content/ | content/ | content/ |
| Glass | molar % | molar % | molar % |
| compositions | (weight %) | (weight %) | (weight %) |
| B60—0.5Ag | 60 (64.2) | 39.5 (34.0) | 0.5 (1.8) |
| B60—1Ag | 60 (63.3) | 39 (33.1) | 1 (3.5) |

In certain embodiments of any of the foregoing or following, whether doped with silver or not, the composition has a higher borate content than the content of any of the other components individually. For example, the borate content is more than the calcium content, or the doping component.

In certain embodiments of any of the foregoing or following, whether doped with silver or not, the composition has borate as its sole and/or main network forming component. This is distinct from glasses containing boron where borate is not the sole and/or major network former such as in borophosphate, borosilicate, aluminoborate and aluminoborosilicate glasses.

In certain embodiments of any of the foregoing or following, whether doped with silver or not, the compositions have calcium as the main component of the glass system, or as the most abundant component. The calcium oxide content can be higher than any of the other components individually.

In certain embodiments of any of the foregoing or following, whether doped with silver or not, the compositions have borate and calcium which can both act as network formers, or one can act as the network former and the other as the modifier.

### Borate-Magnesium Glasses

According to certain embodiments of the present technology, glass compositions have two components: a borate component and a magnesium oxide component. In these binary compositions, xB₂O₃.yMgO, x is from about 30-80 mol%, and y is from about 20-70 mol%. In certain other embodiments, x is from about 20-90 mol% and y is from about 10-80 mol%.

Examples of the two component borate glasses including a borate component and a magnesium oxide component are provided in Table 3 below:

TABLE 3
Example borate and magnesium oxide based glass compositions

| | | Magnesium oxide |
|---|---|---|
| | Borate (B₂O₃) | (MgO) component |
| | component content/ | content/molar % |
| Glass composition | molar % (weight %) | (weight %) |
| B80Mg | 80 (87.4) | 20 (12.6) |
| B70Mg | 70 (80.1) | 30 (19.9) |
| B60Mg | 60 (72.2) | 40 (27.8) |
| B50Mg | 50 (63.3) | 50 (36.7) |
| B40Mg | 40 (52.5) | 60 (46.5) |
| B30Mg | 30 (42.6) | 70 (57.4) |

In certain embodiments, these two component systems including a doping component. The doping component can be trace amounts. Alternatively, the doping component can be more than a trace amount. The doping component can be any one or more of silver, titanium, lithium, silicon, gold, copper, cobalt, fluoride, iron, manganese, molybdenum, magnesium, nickel, rubidium, strontium, potassium, zinc, niobium, cesium, and gallium. In certain embodiments of any of the foregoing or following, the doping component comprises about 0.05 to about 10 wt. % or about 0.05 to about 30 wt. % of the composition. The content of silver oxide as the doping agent is determined by potential toxic levels. In certain embodiments, the content of the doping agent is about 0.1-10 wt %.

Table 4 shows example borate-magnesium oxide compositions including silver oxide as a doping component.

TABLE 4
Example borate-magnesium oxide compositions including silver
as a doping agent

| | Borate | Magnesium | |
|---|---|---|---|
| | (B₂O₃) | oxide (MgO) | |
| | component | component | Silver oxide |
| | content/ | content/ | (Ag₂O) doping |
| Glass | molar % | molar % | content/molar % |
| compositions | (weight %) | (weight %) | (weight %) |
| B60Mg—0.5Ag | 60 (71) | 39.5 (27) | 0.5 (2) |
| B60Mg—1Ag | 60 (69.8) | 39 (26.3) | 1 (3.9) |

The sol-gel derived compositions have a higher surface area than compositions made by melt-quench derived methods. Advantageously, in certain embodiments, surface area of certain embodiments of the present biomaterials results in faster ionic release and degradation which may improve bioactivity by affecting cellular response and promoting quicker tissue regeneration.

In certain embodiments of any of the foregoing or following, whether doped with silver or not, the compositions have magnesium as the main component of the glass system, or as the most abundant component. The magnesium oxide content can be higher than any of the other components individually.

### Silica-Free, Sodium-Free, Phosphate-Free Compositions

In certain embodiments the compositions are substantially free of one or more of silica, sodium, and phosphate. In other words, the compositions of the present technology do not have silica, sodium, or phosphate-based components formed. In certain embodiments of any of the foregoing or following, the biomaterial/composition is substantially free of alumina. By substantially free is meant that there are no amounts more than possibly trace amounts. Trace amounts of one or more of silica, sodium, and phosphate may be present. In certain embodiments, the silica-free compositions are fully biodegradable.

### Forms of the Composition

The compositions may be in any form. In certain embodiments, the compositions are in particulate form. Particle diameters may range between 0.2-1 μm, 5-2000 μm, 5-100 μm, or 25-75 μm, or any other size which is biologically relevant. In certain other embodiments, the compositions are in another type of solid form (e.g. coating etc.) or in a liquid or gel form.

In certain embodiments, compositions of the present technology in the solid phase have surface areas higher than an equivalent composition made by melt-quench methods. In certain embodiments, the compositions have surface areas per mass of more than: about 1 m²/g, more than about 1 m²/g, about 5 m²/g; more than about 10 m²/g, more than about 20 m²/g, more than about 30 m²/g, more than about 40 m²/g, more than about 50 m²/g; about 5-300 m²/g, 10-300 m²/g, 20-300 m²/g, 30-300 m²/g, 40-300 m²/g, 50-300 m²/g, 60-300 m²/g, 70-300 m²/g, 80-300 m²/g, 90-300 m²/g, 100-300 m²/g, 110-300 m²/g, 120-300 m²/g, 130-300 m²/g, 140-300 m²/g, 150-300 m²/g, 200-300 m²/g, 250-300 m²/g, 5-250 m²/g, 5-200 m²/g, 5-150 m²/g or 5-100 m²/g.

In certain embodiments, the compositions have pore volumes per mass of: more than about 0.001 cm³/g, more than about 0.01 cm³/g, more than about 0.02 cm³/g, more than about 0.03 cm³/g, more than about 0.04 cm³/g, more than about 0.05 cm³/g, more than about 0.06 cm³/g, more than about 0.07 cm³/g, more than about 0.08 cm³/g, more than about 0.09 cm³/g, more than about 0.1 cm³/g, more than about 0.2 cm³/g, more than about 0.3 cm³/g, more than about 0.4 cm³/g; between about 0.1-3.0 cm³/g, 0.2-3.0 cm³/g, 0.3-3.0 cm³/g, 0.4-3.0 cm³/g, 0.5-3.0 cm³/g, 0.6-3.0 cm³/g, 0.7-3.0 cm³/g, 0.8-3.0 cm³/g, 0.9-3.0 cm³/g, 1.0-3.0 cm³/g, 0.1-2.5 cm³/g, 0.42-1.18 cm³/g or 0.1-2.0 cm³/g.

All surface area per mass measurements are as measured using the isotherm with the Brunauer-Emmett-Teller (BET) method (S. Brunauer, P. H. Emmett, E. Teller, Adsorption of gases in multimolecular layers. Journal of the American Chemical Society 60, 309-319 (1938)).

Methods of Making the Compositions

Aspects of the present technology include methods of making the compositions of the present technology. Present methods include an adapted sol-gel method. Sol-gel methods offer a range of advantages over traditional melt-quench derived methods of making glasses, including lower processing temperatures providing more compositional choice, higher surface area and porosity meaning higher rates of dissolution and bioreactivity, controllable surface area and porosity meaning controllable bioactivity.

Briefly, certain embodiments of the method comprise forming a solution using precursors of the composition, gelling the solution to form a gel of the composition, drying the gel to form a dry gel, calcining the dry gel to remove organic matter, and optionally grinding the calcined dry gel, and/or sizing the calcined dry gel to obtain particles within a certain size and/or shape range. For the two-component borate-calcium oxide compositions, certain methods for making the composition comprise: combining precursor solutions containing boron ions, and calcium ions to form a solution; gelling the solution to form a gel; drying the gel; and calcining the dried gel. For the two-component borate-magnesium oxide compositions, certain methods for making the composition comprise: combining precursor solutions containing boron ions, and magnesium ions to form a solution; gelling the solution to form a gel; drying the gel; and calcining the dried gel.

### Precursors

In certain embodiments, forming the solution comprises mixing together precursors. Example precursors are set out below:

For the borate component: Boric acid H₃BO₃, Trimethyl borate B(OCH₃)₃, triethyl borate B(C₂H₅O)₃, tributyl borate B(CH₃(CH₂)₃O)₃, Tri-tert-butyl borate (B₃(CH₃)₃CO). Preferably, the boron ion precursor solution is boric acid which may be dissolved in ethanol or methanol.

For the calcium oxide component: Calcium nitrate tetrahydrate (Ca(NO₃)₂4H₂O), Calcium Chloride (CaCl₂), Calcium Ethoxide (Ca(C₂H₅O)₂), Calcium methoxide (C₂H₆CaO₂), Calcium methoxyethoxide 5-40% but preferably 20% in methoxyethanol (C₆H₁₄CaO₄), Calcium citrate (Ca₃(C₆H₅O₇)₂), Calcium citrate tetrahydrate (C₁₂H₁₈Ca₃O₁₈), Calcium citrate malate (C₆H₇O₇)ₓ·(C₄H₅O₅)_{Y}·(Ca₂+)_{z}), Calcium lactate (C₆H₁₀CaO₆), Calcium lactate monohydrate (C₆H₁₂CaO₇), Calcium lactate pentahydrate (C₆H₂₀CaO₁₁), Calcium lactate trihydrate (C₆H₁₆CaO₉), Calcium lactate gluconate (C₉H₁₆CaO₁₀), Calcium sulfate (CaSO₄), Calcium sulfate dihydrate (CaH₄O₆S), Calcium sulfate hemihydrate (CaSO₄·0.5H₂O), Casein phosphopeptide-amorphous calcium phosphate (CaₓH_{y}(PO₄)_{z}·nH₂O where n is between 3 and 4.5), Calcium acetate (C₄H₆O₄Ca), Calcium carbonate (CaCO₃).

For the magnesium oxide component: Magnesium methoxide (C₂H₅MgO₂), Magnesium ethoxide (C₄H₁₀MgO₂), Magnesium methoxide 5-20% but preferably 7-8% in methanol (C₂H₆MgO₂), Magnesium methoxyethoxide 5-40% but preferably 25% in methoxyethanol (C₆H₁₄MgO₄), Magnesium Lactate, trihydrate (C₆H₁₀MgO₆·3H₂O), Magnesium nitrate (Mg(NO₃)₂), Magnesium nitrate dihydrate (Mg(NO₃)₂·2H₂O), Magnesium nitrate hexahydrate (Mg(NO₃)₂·6 H₂O), Magnesium acetate (Mg(CH₃COO)₂), Magnesum acetate tetrahydrate (C₄H₁₄MgO₈).

In certain embodiments, the precursor solutions for borate-calcium oxide two-component glass compositions comprise boric acid, ethanol and/or methanol, and calcium methoxyethoxide (20% in methoxyethanol).

In certain embodiments, the precursor solutions for borate-magnesium oxide two-component glass compositions comprise boric acid and ethanol and Magnesium methoxyethoxide 25% in methoxyethanol.

Advantageously, certain embodiments of the present method do not require the addition of water. In contrast, in traditional sol-gel methods, water is added to induce hydrolysis of the precursor materials and to allow for condensation creating the initial sol. In certain embodiments of the present method, boric acid is added to ethanol which creates triethyl borate (TEB) and water as seen in equation 1.

H₃BO₃+3(C₂H₆OH)→B(C₂H₅O)₃+3(H₂O) (eqn. 1)

It is believed that excess water formed by this reaction hydrolyzes the newly formed TEB creating terminal OH⁻ groups which undergo condensation forming the initial sol and eventually the gel network. These processes can also happen at the same time. The calcium and/or magnesium sources incorporate themselves into the network during processing.

### Mixing the Precursors

In certain embodiments of any of the foregoing or following, the pH of the solution after adding the final precursor is, or is adjusted to, more than about 10, more than about 10.5, more than about 11, more than about 11.5, more than about 12, or more than about 12.5. In certain embodiments of any of the foregoing or following, the pH of the solution after adding the final precursor is between about 10.5 and about 14.0, or about 11 and about 13.5.

In certain embodiments, the boric acid is dissolved in ethanol. In certain embodiments, the boric acid is dissolved in ethanol at a temperature at or higher than room temperature, at temperatures between about room temperature and 40° C., at about, 39° C., at about 38° C., at about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., or about 21° C.

Advantageously, boric acid has a higher solubility in ethanol at higher temperatures which means that when the mixing step is carried out at 37° C., for example, less ethanol is required to dissolve the boric acid.

In certain embodiments, the boric acid is dissolved in methanol. In certain embodiments, the boric acid is dissolved in methanol at a temperature at or higher than room temperature, at temperatures between about room temperature and 40° C., at about, 39° C., at about 38° C. at about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., or about 21° C.

Advantageously, boric acid has a higher solubility in methanol compared to ethanol, and at higher temperatures which means that when the mixing step is carried out at 37° C., for example, less methanol is required to dissolve the boric acid.

In certain embodiments of any of the foregoing or following, the precursor solutions are added sequentially. In certain embodiments of the foregoing or following, the precursor solutions having a basic pH are added after those with a low pH.

In certain embodiments, the mixing of the precursor solutions is performed in a single pot. Advantageously, this means that this process can be easily scaled-up.

In other examples, boric acid and methanol are mixed in one container then Ca-lactate-5H₂O and methanol are mixed in a separate container. When each solution becomes clear, they are then added together to form the final sol (all at room temperature). The sol is then cast into a vial and gelation occurs (e.g. a clear gel is formed) within 3 days.

In certain embodiments of any of the foregoing or following, a pH adjusting agent can be used to adjust the pH of the solution after mixing the precursors. The pH adjusting agents can be selected from NaOH, KOH, LiOH, ammonia, calcium hydroxide (Ca(OH)₂), and strontium hydroxide (Sr(OH)₂).

### Gelling

In certain embodiments of the foregoing or following, gelling the solution comprises maintaining the solution at a temperature between about room temperature and about 60° C., preferably at about 37° C. There may also be included a step of ageing the gel comprising allowing it to rest at room temperature or at elevated temperatures. In certain embodiments of any of the foregoing or following, an ageing step is not necessary as gelation may stabilize rapidly.

In certain embodiments of any of the foregoing or following, the gelling step takes place at a temperature of between about room temperature and about 38° C., optionally at about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., or about 21° C.

In certain embodiments of any of the foregoing or following, the gelling step takes place in less than about 168 hours, less than about 72 hours, less than about 24 hours, less than about 12 hours, less than about 10 hours, less than about 8 hours, less than about 6 hours, less than about 4 hours, less than about 2 hours, less than about 1 hour, less than about 30 minutes, or between about 5 minutes and about 30 minutes. Gelation of the solution is considered to have occurred if no flow is observed when a vial containing the solution is held upside down at room temperature and pressure and no flow is observed.

The inventors found that with certain embodiments of the disclosed method and biomaterial, a rapid gelation was observed. In certain embodiments, gelation was achieved within 30 minutes of adding the final precursor. A pH adjustment may be necessary to modulate the speed of gelation. Shorter biomaterial processing times are economically desirable. Advantageously, the sol-gel method presently described is a simple method which can be scaled up.

In certain embodiments of any of the foregoing or following, the gelling step includes allowing the gels to age for example at room temperature or at temperatures higher than room temperature for about 0.5-15 days or until gel formation has stabilized or completed.

### Drying

In certain embodiments of the foregoing or following, drying the gel comprises heating the gel and/or allowing loss of humidity to form a dry gel. Drying can include leaving the gelled solution at room temperature or elevated temperatures for example for about 1-15 days.

In certain embodiments, drying the gels comprises removing from the oven and placing in crystallization dishes to dry at room temperature for a day then in an oven at 120° C. for 0.2-72 days. In some embodiments, the drying step causes the gel to dry to a particulate form. In certain embodiments of the foregoing or following, the method comprises combining the gel with a polymer or binder and drying under controlled conditions such as those using critical point drying. This may provide a biomaterial with a monolithic structure.

### Calcining

Calcining the dry gel comprises, in certain embodiments, heating the dry gel, such as to between about 400-700° C. This may eliminate organic contamination. Alternatively, calcining the dry gel can comprise heating to between about 100 to about 400° C., or from about 120 to about 400° C., or from about 150 to about 400° C., or from about 200 to about 400° C. Any suitable heating rate, dwell time and cooling rate can be used. In certain embodiments of the foregoing or following, heating comprises using a 3° C/min heating rate, followed by a 2-hour dwell, and then furnace cooling.

Further processing

After calcining, the particles may be further processed, such as to obtain particles of a certain size or shape. The further processing step may include a grinding step, or a sieving step. Particles thus obtained may have a diameter range of 0.2-1 μm, 5-2000 μm, 5-100 μm, or 25-75 μm, or any other size which is biologically relevant.

The particles or the gel obtained by the sol-gel method may be in any form or may be processed to any form, including but not limited to: a fibrillar form, a particulate form, a hollow spherical form, a bead form, a solid spherical form, a conical form, a wedge shaped, a cylindrical form, a film form, a foam form, a sponge form or a monolithic form. Other shapes are also possible, as well as a range of sizes. Biomaterials in the form of thin films are useful as coatings.

In certain embodiments of the foregoing or following, the method comprises a processing step after gelation to make fibres (e.g. by electrospinning, solution blow-spinning etc), thin films, or hollow particles.

### Methods of Doping

In certain embodiments, methods of doping sol-gel derived borate glass compositions, not limited to the borate glass composition of the present technology, comprise an adapted sol-gel method. Broadly, in certain embodiments, the methods of doping differ from the sol-gel methods described above in that a precursor solution including silver ion is added to an acidic precursor solution. The method may include adjusting the pH of the solution, or ensuring that the order of mixing the precursor solutions comprises mixing the precursor including the silver ion with an inherently acidic other precursor solution.

More specifically, in certain embodiments, the method may comprise providing a precursor solution for the borate component (precursors are as noted above, e.g. boric acid and ethanol), providing a precursor solution for the calcium oxide component (precursors are as noted above, e.g. calcium methoxyethoxide), and providing a precursor solution for the silver oxide component. Silver ion precursors may include silver nitrate, silver acetate or silver carbonate. The precursor solutions are mixed such that the silver ion precursor is added to an acidic solution. More specifically, the silver ion solution is added to the ethanol and boric acid mixture (about pH 4), and before the addition of the calcium precursor solution (calcium methoxyethoxide, pH 10). In certain other embodiments, the pH of the mixture may be adapted by the addition of a pH modifying agent, such as an acid.

In certain other embodiments, the method may comprise providing a precursor solution for the borate component (precursors are as noted above, e.g. boric acid), providing a precursor solution for the calcium oxide component (precursors are as noted above, e.g. calcium methoxyethoxide), providing a precursor solution for the silver oxide component (e.g. silver nitrate), and providing a precursor solution for phosphate ions (e.g. triethyl phosphate). In this case, the precursor solution for the silver ion can be added to the boric acid solution, or to the triethyl phosphate solution, which both have a pH of about 4, but before the calcium ion precursor solution.

The method of making the silver doped composition may continue in the same manner as that of the sol-gel methods described above, e.g. including one or more of the gelling, ageing, drying and calcination steps described above.

### Biomaterial Formation

The composition may be further processed to be formed into a biomaterial. The biomaterial may be a slurry, an emulsion, a solution, a gel, a putty or a paste. The biomaterial may also be a coating. The biomaterial can be incorporated into a delivery substrate such as a dressing. In this respect, the biomaterial comprises the composition and an excipient or a carrier. The carrier may be in the form of a slurry, emulsion, solution, gel, putty or paste. The biomaterial may also include additional components such as bioactive agents.

### Carriers/Excipients

In certain embodiments, the carrier is a paste, such as for dental applications such as dentine hypersensitivity. Potential paste compositions include glycerine, PEG400, Klucel, titanium dioxide, and syloid. An example biomaterial including an embodiment of the present composition and a paste as a carrier is: Glycerine (55-70wt %), PEG400 (13-23wt %), Klucel (0-1.5wt %), TiO₂ (0.5-2wt %), Syloid 63 (3-8wt %), glass composition (3-15 vol.%).

The biomaterial may further comprise flavor components, such as artificial or natural flavors, sweeteners, etc. The biomaterial may also include a preservative, such as sodium benzoate, methyl paraben, and ethyl paraben.

In certain other embodiments, the carrier is a synthetic polymer selected from the group consisting of vinyl polymers, polyoxyethylene-polyoxypropylene copolymers, poly(ethylene oxide), acrylamide polymers and derivatives or salts thereof. The vinyl polymer may be selected from the group of polyacrylic acid, polymethacrylic acid, polyvinyl pyrrolidone or polyvinyl alcohol. The carrier may be a carboxy vinyl polymer or a carbomer obtained by polymerisation of acrylic acid. The carrier may be poly(lactic-co-glycolic acid) (PLGA), Polylactic acid or polylactide (PLA), Polycaprolactone (PCL), or any thermoplastic or biodegradable polyester or polymer.

The carrier may be a protein-based polymer selected from at least one of gelatin, collagen, fibrin, silk fibroin, elastin, and the like. Any other cosmetic cream or serum can be used as a carrier for the biomaterial.

The carrier may comprise a polysaccharide selected from at least one of sodium hyaluronate, hyaluronan, starch, chitosan, chitin, agar, alginates, xanthan, carrageenan, guar gum, gellan gum, pectin, locust bean gum, and the like.

The carrier can be a liquid such as blood, water, saline or simulated body fluid.

In certain embodiments, the carrier is a hydrogel.

The carrier may comprise a bone or defect filler material such as polymethylmethacrylate or calcium phosphate-based bone cements.

### Bioactive Agents

The biomaterial may comprise one or more bioactive agents selected from cells, genes, drug molecules, therapeutic agents, particles, osteogenic agents, osteoconductive agents, osteoinductive agents, anti-inflammatory agents, antibiotics, anticoagulants, angiogenic agents, growth factors, and the like. The biomaterial may be used as a delivery vehicle for these bioactive agents.

Examples of cells include those involved in hard and soft tissue generation, regeneration, repair and maintenance, for example embryonic or mesenchymal stem cells, bone marrow stem cell, osteoblasts, preosteoblasts, fibroblasts, nerve cells, muscle cells, myoblasts, fibroblasts, populations of cells such as from a bone marrow aspirate, chondrocytes, and the like. Combinations of cell types can also be included. Therapeutic agents can include hormones, bone morphogenic proteins, antimicrobials, anti-rejection agents and the like. Examples of drugs include any molecules for disease, condition or symptom treatment or control, anti-inflammatory, growth factors, peptides, antibodies, vesicle for release of ions, release of gas, release of nutrients, enzymes, as well as nano carriers. The particles can be fibroin-derived polypeptides, preferably polypeptides which have been chymotryptically isolated and extracted from silk fibroin such as a soluble fraction Cs, a precipitated fraction Cp, or a combination of the Cs and Cp fractions (as described in PCT/CA2012/000192, the contents of which are herein incorporated by reference).

### Coatings and Substrates

The biomaterial can be in the form of a coating. The coating can be provided on an implant for example, such as a bone implant. The biomaterial can also be coated onto hard tissue such as bone or teeth. In certain embodiments, the biomaterial may be used for bone mass increase for tooth implants, endosseus ridge bone enhancement for denture fixation, as well as maxillofacial and orthopedic uses.

In certain embodiments, the biomaterial is included within a substrate. The substrate can be a sponge, a dressing, a skin patch, etc. Biomedical uses include as a hemostatic material, such as a hemostatic sponge, by virtue of its high surface area. The biomaterial may also be used as a cosmetic for skin for exfoliation and generally improving skin properties.

In certain embodiments, the composition or biomaterial can be incorporated into a polymer matrix, degradable or otherwise, to create bioactive composite systems. The composite can be in a monolithic form, a fibrous form, or a porous sponge scaffold form. The volume fraction of the biomaterial in the composition system can range from about 0.0001 to about 0.8.

### Methods of Mineralization

In certain embodiments, the present technology comprises methods of forming minerals or mineralisation in vitro or in vivo using compositions or biomaterials of the present technology.

By mineralisation is meant that the compositions induce formation of hydroxyapatite and/or calcite through dissolution and ion release in vivo. In some embodiments, the biomaterial can induce bone formation. A representative and accepted in vitro model for in vivo mineralisation behaviour is that of contact with simulated body fluid

Bone formation and mineralisation may comprise apatite formation such as hydroxyapatite formation in the absence or presence of cells.

Applications for such use of the compositions include bone regeneration or augmentation, in which case the composition/biomaterials are placed in a bone defect or at a site requiring bone augmentation. The biomaterial may be made into a slurry using the patient's own blood or bone before packing into the defect. The biomaterial may also be injected.

Other applications include preventing or treating dentine hypersensitivity. In these uses, the present compositions/biomaterials may be applied to the teeth of a user as a paste or as a solution, such as a mouthwash. The treatment may be chair-side or over the counter, at home use.

While there are many different products used to treat dentin hypersensitivity, there are still many inherent drawbacks with existing approaches. Although desensitizing agents, like potassium nitrates, are included in many in-office as well as over the counter toothpastes and mouthwashes, they often require long-term use to be effective and do not treat the main cause of dentin hypersensitivity, but rather numb the problem. The commonly used fluoride varnishes and glutaraldehyde often need to be applied carefully by dental professionals since the ingredients can be hazardous and damage the surrounding soft tissue, thus causing more problems. Furthermore, similar to desensitizing agents, these approaches typically require multiple treatments to be effective and do not help regenerate the lost dentin mineral. Other methods such as laser therapies are relatively new and their method of action and clinical effectiveness still need to be determined. Physical tubule blockage methods using toothpastes incorporating calcium carbonate are commonly applied, however, this mineral does not have the same composition as tooth enamel, and is thus mechanically weaker, making it easier to remove during everyday wear. Moreover, ceramic particles like tricalcium phosphate, have slow dissolution rates compared to bioactive glasses thus limiting the ion release rate and conversion. Bioactive glasses, such as NovaMin® and BioMin™, which have been incorporated into toothpastes, can release calcium and phosphate ions, but due to their processing and silicate-based chemistry, they are unable to undergo full conversion to hydroxyapatite. In contrast, the unique chemistry and processing of the present compositions and biomaterials eliminates the drawbacks of typical bioactive glasses allowing them to undergo rapid and full conversion to hydroxyapatite.

### Non-mineralizing Uses

In certain embodiments, the present technology comprises methods of treating soft tissues in vitro or in vivo using compositions or biomaterials of the present technology. In certain embodiments, the present technology comprises uses of the compositions or biomaterials of the present technology for treating soft tissues in vitro or in vivo.

Such soft tissues treatments include supporting direct axon growth, wound healing, protecting, regenerating or repairing soft tissues including for both medical and cosmetic applications. Examples of soft tissue include vascularization, wound healing, cartilage, skin, muscle, tendon, ligament, cornea, iris, periodontal tissue, bladder, cardiac, lung, nerve, gastrointestinal, urinary tract and laryngeal tissue repair.

There is also provided use of the biomaterial described herein for drug or other bioactive agent delivery, as a hemostatic agent, as an anti-cancer agent, and for antimicrobial use.

There is also provided use of the biomaterial as described herein as a cosmetic for skin for exfoliation, for improving skin properties, for reducing skin redness, for reducing the appearance of wrinkles, for reducing the appearance of ageing, or for skin oxidative properties. The biomaterial may be included within a cream or a paste for application to skin.

Uses of the composition include filling hard or soft tissue defects, as a coating on a bone implant, as a drug delivery vehicle, or for enhancing the appearance of skin.

Identification of equivalent compositions and methods are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

### Example 1

### Method of Making Sol-Gel Derived Borate-Calcium Oxide Glass Compositions

Compositions, according to certain embodiments of the present technology, were made using a sol-gel process. All sol-gel processing took place in air or within a nitrogen purged glove box. Two component borate glass compositions based on boron and calcium were made by incrementally increasing or decreasing the amount of boron and calcium oxide respectively (Table 1). Boric Acid (>99.5%) and anhydrous ethanol were mixed and heated to about 35-50° C., preferably 40° C±3° C., to aid dissolution. Advantageously, unlike most known sol-gel methods, a mild heating of about 35-50° C. suffices. Once the solution became clear, calcium methoxyethoxide (20% in methoxyethanol) was added in a drop wise manner. After the final addition the solution was mixed for another 30 minutes or until gelation occurred (as determined by the viscosity becoming too great for stirring). The solution was then cast into vials, sealed, and stored at 37° C. for ten days for further gelation and ageing. The gels were then transferred to crystallization dishes and dried in air at room temperature for one day, followed by oven drying at 120° C. for 2 days. The particles were then calcined in air (400° C.) using a 3° C/min heating rate, 2-hour dwell time, and then furnace cooled. Lower calcination temperatures (100° C. to 400° C.) are also possible. Following calcination, the particles were ground and sieved to isolate a particle size fraction of 25-75 μm and stored in a desiccator until analysis.

The specific surface areas of the calcined powders (400° C., n=3) were measured with N₂ (g) adsorption and desorption isotherms collected with a Micrometics TriStar 3000™ (Micromeritics Instrument Corporation, USA) gas sorption system. The specific surface areas were determined from the isotherm with the Brunauer-Emmett-Teller (BET) method (S. Brunauer, P. H. Emmett, E. Teller, Adsorption of gases in multimolecular layers. Journal of the American Chemical Society 60, 309-319 (1938)). The average pore width and pore volume was provided using the adsorption isotherms using the Barrett-Joyner-Halenda (BJH) method (L. G. Joyner, E. P. Barrett, R. Skold, The Determination of Pore Volume and Area Distributions in Porous Substances. II. Comparison between Nitrogen Isotherm and Mercury Porosimeter Methods. Journal of the American Chemical Society 73, 3155-3158 (1951); published online Epub1951/07/01 (10.1021/ja01151a046)). The results are presented in Table 5.

TABLE 5
Glass particle textural properties: Average Median (D50) and Mean (DAVG) Diameter,
Specific Surface Area (SSA), Average Pore Width, Average Pore Volume, and density

| | Partide Size (μm) | | SSA | Pore Volume | Pore Width | Density |
|---|---|---|---|---|---|---|
| ID | D₅₀ | D_{AVG} | (m²/g) | (cm³/g) | (nm) | (g/cm³) |
| B30 | 30.5 | 37.8 | 66 ± 1 | 0.37 ± 0.01 | 16.0 ± 0.5 | 2.64 |
| B40 | 30.8 | 39.2 | 77 ± 2 | 0.44 ± 0.02 | 16.3 ± 0.5 | 2.49 |
| B50 | 26.4 | 34.5 | 107 ± 5 | 0.87 ± 0.09 | 23.6 ± 0.5 | 2.42 |
| B60 | 22.0 | 29.7 | 158 ± 17 | 1.22 ± 300.13 | 20.3 ± 0.9 | 2.28 |
| B70 | 21.1 | 28.4 | 91 ± 4 | 0.63 ± 0.04 | 20.9 ± 0.4 | 2.18 |
| B80 | 51.0 | 54.9 | 1 ± 0 | 0.07 ± 0.01 | 84.0 ± 0.9 | 1.78 |

Specific surface area and porosity remained high for all compositions except for B80. Density showed a decreasing trend with increasing B₂O₃.

### Example 2

### Structure of the Compositions of Example 1

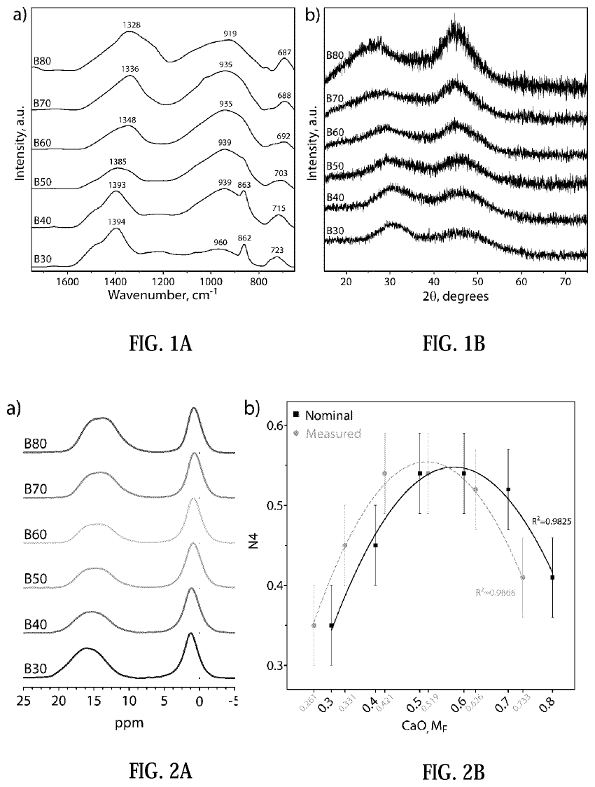

FIGS. 1A and 1B illustrate the Attenuated total reflectance-Fourier transform infrared (ATR-FTIR) spectra and x-ray diffraction diffractograms, respectively, for the compositions of Example 1. For the ATR-FTIR, a Spectrum 400™ (Perkin-Elmer) was used to collect spectra in a wavenumber range between 4000 and 650 cm⁻¹ with a resolution of 4 cm⁻¹ using 64 scans per sample. All spectra were baseline corrected and normalized to the total area surface area under absorption bands using Spectrum software (Perkin-Elmer, USA).

The three main regions associated with borate-based glasses were present at 850-1200 cm⁻¹ B—O stretching of BO₄⁻units), 1200-1500 cm⁻(B—O stretching of BO₃ units), and a band at ˜720 cm⁻attributable to the B—O—B bending of BO₃³⁻ units, which was more defined in lower borate content glasses. At lower CaO amounts it can be seen that peaks relating to the B—O stretching units are smaller. With increasing CaO content, these peaks become larger and suggesting more 4-coordinated units. However, with further increasing CaO content these properties begin to reverse and the B—O stretching units become smaller. This trend is due to the borate anomaly.

The XRD diffractograms show that all the compositions of Example 1 were amorphous at a 400° C. calcination temperature which suggests homogeneity. ATR-FTIR shows 3- and 4-coordinated borate units.

### Example 3

### Controlling Structure in the Compositions

FIGS. 2A and 2B illustrate the NMR stack plots and B4 coordination, respectively, of the compositions of Example 1, using solid state ¹¹B nuclear magnetic resonance (NMR). The ¹¹B magic angle spinning (MAS) NMR experiments were carried out on a Bruker Advanced NMR spectrometer in the NMR-3 at Dalhousie University with a 16.4 T magnet (224.67 MHz ¹¹B Larmor frequency) using a probe head for rotors of 2.5 mm diameter. The samples were spun at 10 and 25 kHz to determine center bands and to identify spinning sidebands. In addition, a spectrum of an empty rotor was acquired under identical conditions. The NaBH₄ resonance served as secondary chemical shift standard at −42.1 ppm relative to BF₃·Et₂O. The ¹¹B NMR spectra were accumulated using a single pulse with pulse length of 0.56 μs corresponding to a 15-degree pulse angle in the nearly cubic environment of NaBH₄. The small pulse angles were chosen to allow the comparison of sites with different quadrupole couplings. Rough spin lattice relaxation times were determined using a saturation recovery sequence. The pulse repetition times were chosen to be on the order of the longest relaxation time, which varied between 4 and 25 seconds. Between 80-160 scans were accumulated varying with the boron concentration. The substantial boron background was removed by subtracting the spectrum of an empty rotor acquired under similar conditions having accumulated 320 scans. The integral values are given between 23.0 to 6.0 ppm and from 6.0 ppm to −5.0 ppm.

¹¹B MAS NMR spectroscopy provided geometrical information on the borate unit of the compositions of Example 1. All biomaterials exhibited a large, fairly sharp peak near 0 ppm which can be attributed to ¹¹B nuclei occupying a fairly symmetric site in chemical structure.

The small broadening to the left of this peak is be due to ¹¹B in less symmetric sites. B30 and B80 showed greater broadening which indicates ¹¹B in asymmetric sites (i.e. BO₃) or the same amount but in sites of less symmetry. The resonance associated with BO₄ was quite narrow located in a chemical shift range around 0 ppm due to its small quadrupole coupling constant Further, when ¹¹B occupies a state of low symmetry, the relaxation times are relatively short (100 ms) due to its quadrupolar nucleus and were likely tetrahedrally coordinated, supporting the ATR-FTIR spectra.

Typical B4 bonding regions are seen around 0ppm while the intensity of the B3 regions around 15 ppm varies composition (FIG. 2A). The boron anomaly is illustrated by the varying calcium oxide content (FIG. 2B). This illustrates that the structure of the borate in the sol-gel derived glass, between a 3 and 4-coordination, can be controlled by adjusting the relative content of the borate and calcium oxide components. While this phenomenon has been well studied, to the inventors' knowledge, this is the first time that this has been demonstrated for a sol-gel derived borate glass and the first time that textural properties (e.g. specific surface area and pore volume) have been shown to follow the borate anomaly as well (TABLE 5).

### Example 4

### Bioactivity—pH Change and Ion Release

Ion release of boron and calcium from the compositions of Example 1 was studied. Glasses (n=3) were submerged in Kokubo's simulated body fluid (pH 7.4) at a 1.5 mg/mL ratio for 0.5, 2, 6, 24, and 168 hours. At the end of each time point the pH was measured. Also the amounts of the calcium ions released into the SBF was quantified using an inductively coupled plasma—optical emission spectrophotometer (ICP-OES). 10 mL aliquots were filtered through a 0.2 μm nylon filter and stored in a 15 mL falcon tube to which 4% (w/v) nitric acid was added. NIST standards were used as calibration values. The pH of the solution was measured at each time point using an Accumet XL20 pH meter.

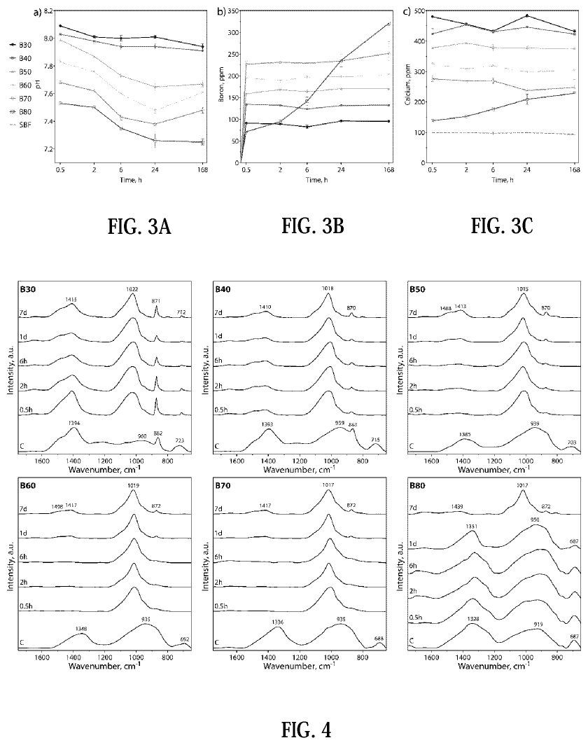

ICP-OES measurements up to 168 hours in SBF revealed the rapid release of boron and calcium ions in the first 30 mins (FIGS. 3B and 3C). The rates and extents of ion release were composition specific showing that ion release could be controlled by compositional modifications. pH increase was also rapid in the first 30 minutes, from the baseline simulated body fluid pH of 7.4, and had a decreasing rate of pH with time after that (FIG. 3A which uses a log scale). Glass with lower specific surface area, B80, showed a more gradual boron ion release over the course of 7 days while the other compositions released the majority of their ions by 30 min as seen by the almost static trend over time. The calcium release rates followed a similar pattern.

### Example 5

### Bioactivity—in Vitro Mineralization

The extent of mineralization of the compositions of Example 1 using Kokubo's simulated body fluid (SBF) (pH 7.4) was investigated. The use of SBF to examine bioactivity is regarded as the standard method to examine acellular mineralization. Glass powder was added to sterile 50 mL falcon tubes containing SBF at a 1.5 mg/mL ratio and stored in an oven at 37° C±1° C. Twice per day, the vials were gently agitated in order to reduce agglomeration of the particles. Mineralization of the glasses was examined at the end of 30 minutes, 2 hours, 6 hours, 1 d, and 7 d time points when the powders were gently rinsed twice with distilled water then twice with ethanol, dried overnight at room temperature, and then dried in an oven for 1 d at 60° C. At each time point the pH of the SBF solution was measured using an Accumet XL20 pH meter (Fisher Scientific).

ATR-FTIR confirmed mineralization was initiated after immersion in SBF in as little as 30 minutes (FIG. 4). The strong band at ˜1018 cm⁻¹ along with shoulders at ˜961 and 1062 cm −¹, are characteristic of the bending modes v1 and v3 of P0₄³⁻, respectively. The broad bands at ˜1470 cm⁻¹ and ˜1421 cm⁻¹ are characteristic of the stretching mode (v1) and (v3) of CO₃²⁻, respectively. The weak band at ˜1640 cm⁻¹ is due to the bending mode (v2) of water. The sharp peak at ˜870 cm⁻¹ indicates the bending mode (v2) of CO₃²⁻as traditionally seen in carbonated apatites.

B30 shows typical phosphate and calcite peaks within 30 min. B40-70 demonstrate rapid surface transformation as seen by the phosphate peaks at 30 min. With longer immersion times these peaks become more defined and along with a more defined carbonate peak seen at 7 d. B80 does not show significant transformation until day 4 where typical hydroxycarbonated apatite (HCA) peaks are observed.

XRD analysis further confirmed calcite formation for B30 and B40 in as little as 30 minutes (Joint Committee on Powder Diffraction Standards JCPDS 5-586). B50, B60, and B70 showed apatite formation in 2 hours, while B80 showed apatite formation within 7 days. (FIG. 5) as indicated by the appearance of peaks at ˜25 and ˜32° 2θ which are characteristic of hydroxycarbonated apatite formation ((JCPDS 19-272), For all compositions, these peaks became more defined with time suggesting increased mineral formation.

B30 and B40 compositions rapidly converted to calcite within 30 min. With longer submersion in the SBF, the B40 composition began to form hydroxycarbonated apatite (HCA) peaks which begin at day 1 and become more defined at day 7. At day 7, B30 also showed slight hydroxyapatite formation. B80 did not show any sign of conversion until day 7.

In summary, the onset mineralization of all the borate-calcium oxide compositions occurred within 30 minutes for B30 and B40 and 2 hours for B50, B60, and B70 hours in SBF. he ability of the compositions to rapidly convert to bone-like hydroxyapatite holds promise for the repair and augmentation of mineralized tissues, such as teeth and bones.

### Example 6

### Scanning Electron Microscopy

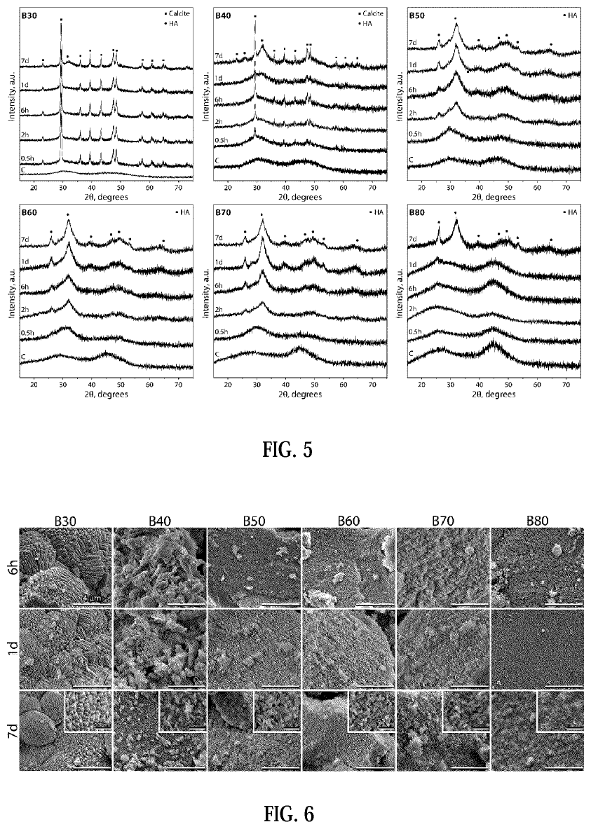

Scanning electron microscopy (SEM) was used to investigate the morphological properties of the glass powders of Example 1. Samples were sputter coated with Au/Pd and analysis was performed with an Inspect F50 Field Emission Scanning Electron Microscope (FEI Corporation, USA) at 10kV. FIG. 6 shows SEM micrographs of the glasses of Example 1. The scale bar represents 10 μm and the inset scale bar represents 1 um. Attributable to sol-gel processing, the surfaces of calcined glasses exhibited a rough, nanoporous texture; corroborating the textural properties in Table 5. Lower borate containing glasses converted to calcite as seen by the typical geometric crystal patterns while increasing borate content led to conversion to hydroxyapatite as seen by the flower-like crystals. B30 and B40 showed signs of both of these crystal formations.

### Example 7

### Cell Viability

Cell culture and Glass Treatment

Human dental pulp stem cells (hDPSCs) were cultured in Dulbecco's Modified Eagle Medium (DMEM) supplemented with 10% fetal bovine serum (FBS) and 1% penicillin streptomycin. Cells were incubated at 37° C. under humidified air containing 5% CO₂. At 80% confluency, the cells were washed with sterile phosphate buffered saline (PBS), detached with 0.25% trypsin ethylenediaminetetraacetic acid solution, and counted using a hemocytometer. Cells between passages 3 and 4 were used for cellular studies.

### Measurement of Cell Metabolic Activity

The AlamarBlue™ assay (Invitrogen) was used to assess the effect of ionic dissolution products on the metabolic activities of hDPSCs. Cells were plated directly into 24-well assay plates at a density of 64000 cells/well and cultured in the presence of ionic dissolution release products generated from the dissolution of the borate glasses in DMEM at two concentrations (0.375 and 0.75 mg/mL). At days 1, 4, and 7 in culture the AlamarBlue™ reagent (5%) was added to each well and, after 4 h incubation at 37° C., 100 mL aliquots were collected and transferred to a 96-well plate for analysis. The fluorescent intensity of reduced AlamarBlue was measured using a Mitras LB 940 microplate reader (Berthold Technologies) equipped with a 555 nm excitation filter and a 580 nm emission filter. After measurements had been completed, media in the 24-well plates was replaced for ongoing treatment. All conditions were tested in triplicate.

### Live/Dead Assay

hDPSCs were plated directly into 96-well assay plates at a density of 16000 cells/well and cultured in the presence of ionic release products generated from the dissolution of borate glasses at two concentrations in DMEM (0.375 and 0.75 mg/mL). After 1, 4, or 7 days in culture, cells were stained with 1 μM calcein-AM and 2 μM ethidium homodimer-1 (Live/Dead assay; Invitrogen) for 15 min. Images of green fluorescent viable cells and red fluorescent dead cell nuclei were acquired in the same well plates using an Olympus IX81 inverted microscope equipped with a UPlanSApo 10 objective (UIS2 series). All conditions were tested in triplicate.

### Measurement of Cell Viability

hDPSCs were plated directly into 96-well assay plates at a density of 16000 cells/well and cultured in the presence of ionic release products generated from the dissolution of the Borate glasses in DMEM at two concentrations (0.375 and 0.75 mg/mL). After 1, 4, or 7 days in culture, cells were incubated with 2 μM calcein-AM in 100 mL PBS. After 15 min, fluorescence was measured in the Mitras LB 940 microplate reader using a 485/535 nm excitation/emission filter pair. All conditions were tested in triplicate.

FIGS. 7A and 7B show cell metabolic activity at 0.375 mg/mL and 0.75 mg/mL, respectively. Metabolic activity was calculated in percent relative to control at day 1. There was an increasing trend in the proliferation rates of cells from day 1 to 7, independent of the condition. It can be postulated that there is no toxic or inhibitory effect of these borate glasses at these concentrations.

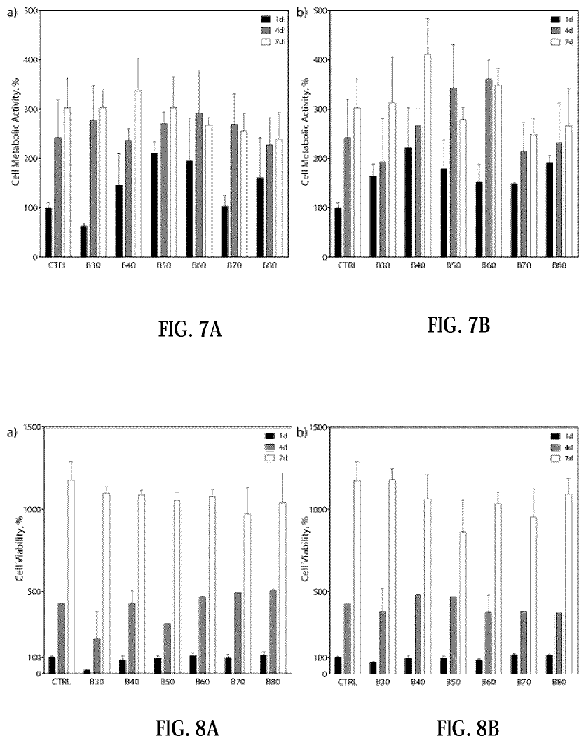

FIGS. 8A and 8B show cell viability at 0.375 mg/mL and 0.75 mg/mL, respectively. Viability was determined in percent relative to control at day 1 from the fluorescent signal of calcein-AM labeled live cells. There were no differences between the borate glass compositions and the control.

The Calcein-AM labelling indicated that the number of viable cells increased over time. No visible dead cells were observed. There were not any significant differences in viability compared to the control.

### Example 8

### Compositions with Silver Doping Component

Sol-gel derived glass compositions according to an embodiment of the present technology was made: 60B₂O₃-(40-X)CaO-XAg₂O where X=0.0, 0.5 and 1 (mol%). Boric acid, calcium methoxyethoxide, and silver nitrate were used as precursors. Boric acid was mixed with ethanol based on the solubility of boric acid (11.2%) into a Teflon beaker covered by a Teflon cap and magnetically stirred at ˜40° C. for 30 min followed by the addition of silver nitrate into the resultant sol and stirred for another 30 min. Calcium methoxyethoxide was then added and the sol were stirred for a final 30 min. Afterwards, the sol was transferred into vials and aged for 5 days at 37° C. The aged sols were initially dried in a fume hood at room temperature for 2 days while covered with a non-transparent box to protect them from exposure to light (since silver can undergo photo-reduction of Ag ions into Ag metal) and then followed by further drying at 120° C. for 2 days. The dried as-made powders were then calcined at 400° C. for 2 h. All glass particles were ground and sieved to 25-75 μm particle size fraction. Table 2 provides a summary of glass compositions investigated in this study.

### Particle Characterization

The average particle size (D_{avg}) and median diameter (D₅₀) of the sieved glass particles was determined using a Horiba LA-920 (ATS Scientific Ink., Canada). The specific surface area (SSA) the particles sieved to 25-75 μm was measured (n=3) with nitrogen gas adsorption and desorption isotherms collected with a Micromeritics TriStar 3000 (Micromeritics Instrument Corporation, USA) gas sorption system. SSA values were determined using the Brunauer-Emmett-Teller (BET) method while the Barrett-Joyner-Halenda (BJH) method [11], using the desorption isotherms, provided the average pore width and pore volume. The results are given in Table 6.

### X-Ray Diffraction

X-ray diffraction (XRD) diffractograms of the glasses were analyzed with a Bruker D8 Discover X-ray diffractometer equipped with a CuKa (λ=0.15406 nm) target set to a power level of 40 mV and 40 mA. Using an area detector, three frames of 25° were collected from 15-75 2 theta)(° and merged in post processing. Phase identification was carried out using X'Pert Highscore Plus.

### Attenuated Total Reflectance-Fourier Transform Infrared Spectroscopy

Attenuated total reflectance-Fourier transform infrared (ATR-FTIR) spectroscopy was carried out using a Spectrum 400 (Perkin-Elmer, USA) between 4000 and 650 cm⁻¹ with a resolution of 4 cm⁻¹ using 64 scans per sample. All spectra were baseline corrected and normalized to the total area surface area under absorption bands using Spectrum software (Perkin-Elmer, USA).

### Inductively Coupled Plasma Optical Emission Spectrometry

Release of silver, boron, and calcium ions from the glass particles in both distilled water (DIW) and simulated body fluid (SBF) at a 1.5 mg/mL ratio, were quantified using an inductively coupled plasma-optical emission spectrophotometer (ICP-OES, Thermo Scientific iCAP 6500, USA). Aliquots were filtered through a 0.2 μm nylon filter and stored in a 15 mL falcon tube to which 4% (w/v) nitric acid (Fisher Scientific, Canada) was added.

Bacteria Growth Curves

Bacteria growth curves were measured by directly exposing the glass particles to Escherichia coli (E.coli) bacteria suspensions in Mueller Hinton Broth 2 (MHB_II, Oxoid; Fisher Scientific Canada) (1.5 and 0. 75 mg/ml) with the initial bacteria concentration of 0.05 optical density (OD) value at 600 nm. Then their OD value was measured up to 24 hours at 37° C. using Tecan Infinite M2000 microplate reader (Tecan group Ltd., Switzerland). All measurements were done in triplicate.

### Migration Assay

Migration assay was performed using the ibidi Culture-Insert 3 Well. Human keratinocyte (HaCat) cells (270,000 cells) were seeded in each well and cultured in a humidified 37° C/5% CO₂ incubator. After 24 hours, the Culture-Insert was gently removed using sterile tweezers followed by washing the cells with PBS twice. The uniform scratch was observed. Then the culture medium was replaced by glass ionic dissolution products (0.75 mg/mL) for another 24 hours. The cells cultured with fresh DMEM, were regarded as control. The cells were then observed with an inverted microscope (Leica DMI 3000B, Germany), and images were taken with a CCD camera (Leica DFC 420C).

### RESULTS

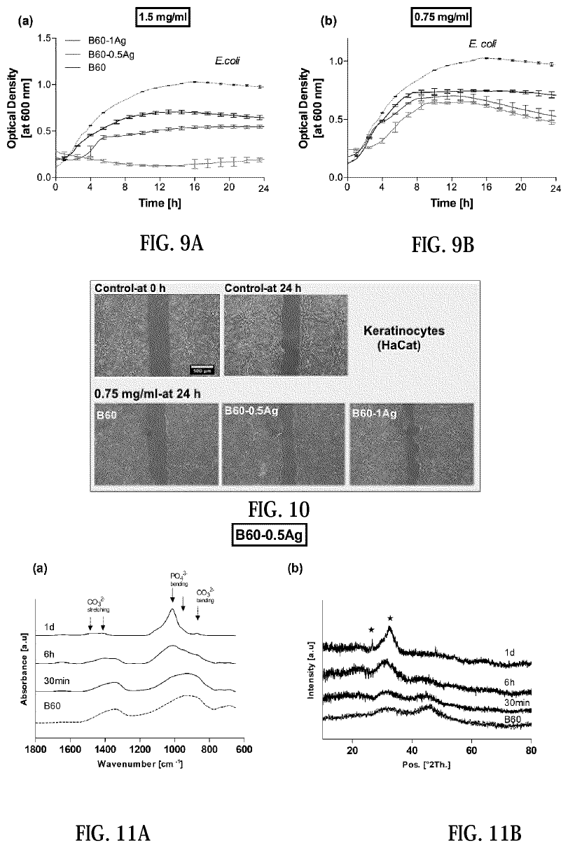

To evaluate the efficacy of the silver-doped compositions of the present technology, glass particles were exposed directly to E.coli bacteria suspension and the growth curve of bacteria was measured. FIGS. 9A and 9B show the growth curves of E.coli when exposed to 1.5 and 0.75 mg/ml of silver doped binary borate compositions of the present technology, respectively. The error bars indicate standard deviation [SD], n=3. It was demonstrated that B60-1Ag glasses were able to stop bacteria production at the 1.5 mg/ml concentration and prevent bacteria growth for 4 h at the 0.75 mg/ml concentration, with lower total bacteria growth at 24 h when compared with E.coli growth curve. Also, B60-0.5Ag glasses were able to delay bacteria production up to 4 h. In general, using glasses at higher glass concentration and/or with higher silver content in the glass composition led to the lower total bacteria growth at 24 h. Additionally, it was observed that non-doped borate glasses at both glass concentrations (1.5 and 0.75 mg/ml) demonstrated a lower total bacterial growth at 24 h when compared with E.coli growth curve. This result suggests non-doped glasses have limited anti-bacterial activity, which can be attributed to their ability to increase the local pH. This result verifies that silver doped compositions of the present technology are highly effective against bacteria associated with wounds resulting in dose dependent efficacy and correlated with silver ion release rates and quantities.

Ionic dissolution products at 0.75 mg/ml glass concentration in control cell culture medium were used to examine the effect of the silver doped compositions on HaCat cell migration (FIG. 10). At 0 h, the scratches were treated with control cell culture medium and ionic dissolution products of the glass particles. After the cells were cultured with control medium and B60, B60-0.5Ag and B60-1Ag for 24 h, the scratch treated with control medium and became slightly narrower, while the scratch treated with the silver doped compositions were significantly recovered when compared to that of the control. The scratch treated with ionic dissolution products of the glass composition with higher silver content (B60-1Ag), had the highest recovery. This indicates the positive effect of silver ions on HaCat cell migration. FIG. 11. Compositions with silver (B6-0.5Ag) still mineralize in SBF within one day according to (a) FTIR and (b) XRD.

TABLE 6
Glass particle textural properties (n = 3): average diameter, specific
surface area, average pore volume and average pore width.

| | | Specific | | |
|---|---|---|---|---|
| | Avg. | surface | Pore | |
| Glass | Diameter | area | volume | Pore width |
| code | (μm) | (m²/g) | (cm³/g) | (Å) |
| B60 | 48 ± 1 | 166 ± 3 | 0.5 ± 0 | 86 ± 0.7 |
| B60—0.5Ag | 50 ± 1 | 180 ± 2 | 0.4 ± 0 | 69 ± 0.4 |
| B60—1Ag | 48 ± 1 | 174 ± 3 | 0.4 ± 0.01 | 65 ± 0.3 |

### Example 9

### Borate-Magnesium Oxide Compositions

A composition ((60)B₂O₃-(40)MgO, mol%) of the present technology comprising a two component system based on borate and magnesium oxide was made by a sol-gel method. Briefly, boric acid (≥99.5%) and anhydrous ethanol (Sigma Aldrich, Canada) were mixed and magnetically stirred in a watch glass-covered Teflon beaker at 40±3° C. to aid dissolution. Once the solution became clear, magnesium methoxyethoxide (25% in methoxyethanol), was added and the sol was mixed for an additional 30 min followed by transferring it to a sealed polypropylene vial, which was then aged at 37° C. for 1 day. The gels were then transferred to crystallization dishes and dried in air at room temperature for 2 days, followed by oven drying at 120° C. for 2 days. Finally, the glass underwent a calcination step at 400° C. at a rate of 3° C/min, with a 2 h dwell, followed by furnace cooling. The calcined glasses were then ground to a particle size fractions of <75 μm and >250 μm.

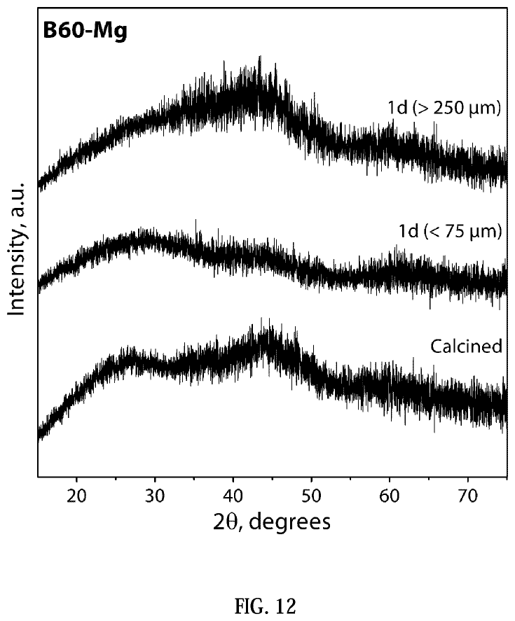

Mineralization of this borate-magnesium composition was investigated using Kokubo's simulated body fluid (SBF) (as previously described in Example 5). X-ray diffraction of both size sets of the particles showed that there was no mineralization of the two-component sol-gel derived borate-magnesium composition after one day immersion in SBF (FIG. 12). Furthermore, the calcined particles were found to be amorphous.

It should be expressly understood that not all technical effects mentioned herein need to be enjoyed in each and every embodiment of the present technology. Modifications and improvements to the above-described implementations of the present technology may become apparent to those skilled in the art. The foregoing description is intended to be exemplary rather than limiting. The scope of the present technology is therefore intended to be limited solely by the scope of the appended claims.

What is claimed is:

1. A composition comprising a sol-gel derived glass, the sol-gel derived glass comprising two main components, the two main components comprising a borate component and an alkaline earth metal component.

2. The composition of claim 1, wherein the alkaline earth metal oxide component is selected from a calcium component, a magnesium component, and a calcium-magnesium component.

3. The composition of claim 1, wherein the sol-gel derived glass is substantially silica free and substantially phosphate free.

4. The composition of claim 1, further comprising a doping component selected from silver, titanium, lithium, gold, copper, cobalt, fluoride, iron, manganese, molybdenum, magnesium, nickel, rubidium, strontium, potassium, zinc, niobium, cesium, and gallium.

5. (canceled)

6. The composition of claim 1, wherein the composition is bioactive, and wherein the alkaline earth metal component is calcium oxide and the composition can undergo mineralization.

7. (canceled)

8. The composition of claim 6, wherein the composition can at least partially convert to hydroxyapatite or calcite, optionally wherein conversion comprises dissolution and precipitation, and optionally wherein a conversion rate ranges from about 30 minutes to about 36 hours, as measured by in vitro testing in simulated body fluid and analyzed by x-ray diffraction.

9. (canceled)

10. The composition of claim 1, wherein the alkaline earth metal component comprises calcium oxide, and the calcium oxide is the main component of the glass system based on weight %.

11. (canceled)

12. The composition of claim 1, wherein the borate component is the main network forming component based on weight %.

13. (canceled)

14. The composition of claim 1, wherein the composition solubilizes to promote wound healing.

15. The composition of claim 1, further comprising a carrier, wherein the carrier is a paste, liquid or gel.

16. The composition of claim 15, wherein the carrier comprises one or more of glycerine, polyethylene glycol, titanium dioxide, and syloid.

17. The composition of claim 1, further comprising a bioactive agent, optionally wherein the bioactive agent is selected from one or more of cells, genes, drug molecules, therapeutic agents, particles, osteogenic agents, osteoconductive agents, osteoinductive agents, anti-inflammatory agents, antibiotics, anticoagulants, and growth factors.

18. The composition of claim 1, wherein the sol-gel derived glass has a surface area per mass of more than: about 1 m²/g, more than about 1 m²/g, more than about 5 m²/g; more than about 10 m²/g, more than about 20 m²/g, more than about 30 m²/g, more than about 40 m²/g, more than about 50 m²/g; about 5-300 m²/g, 10-300 m²/g, 20-300 m²/g, 30-300 m²/g, 40-300 m²/g, 50-300 m²/g, 60-300 m²/g, 70-300 m²/g, 80-300 m²/g, 90-300 m²/g, 100-300 m²/g, 110-300 m²/g, 120-300 m²/g, 130-300 m²/g, 140-300 m²/g, 150-300 m²/g, 200-300 m²/g, 250-300 m²/g, 5-250 m²/g, 5-200 m²/g, 5-150 m²/g or 5-100 m²/g.

19-21. (canceled)

22. A method for making the composition of claim 1, comprising combining precursor solutions containing boron ions, with alkaline earth metal ions to form a solution; gelling the solution to form a gel; drying the gel; and calcining the dried gel.

23. The method of claim 22, wherein the alkaline earth metal ions comprise calcium ions, magnesium ions, or calcium and magnesium ions.

24. The method of claim 22, wherein the precursor solution containing boron ions is selected from trimethyl borate B(OCH₃)₃, triethyl borate B(C₂H₅O)₃, tributyl borate B(CH₃(CH₂)₃O)₃, Tri-tert-butyl borate (B₃(CH₃)₃CO) and boric acid, dissolved methanol or ethanol.

25. The method of claim 22, wherein the precursor solution containing calcium ions is selected from Calcium nitrate tetrahydrate (Ca(NO₃)₂4H₂O), Calcium Chloride (CaCl₂), Calcium Ethoxide (Ca(C₂H₅O)₂), Calcium methoxide (C₂H₆CaO₂), Calcium methoxyethoxide 5-40% but preferably 20% in methoxyethanol (C₆H₁₄CaO₄), Calcium citrate (Ca₃(C₆H₅O₇)₂), Calcium citrate tetrahydrate (C₁₂H₁₈Ca₃O₁₈), Calcium lactate monohydrate (C₆H12CaO₇), Calcium lactate pentahydrate (C₆H₂₀CaO₁₁), Calcium lactate trihydrate (C₆H₁₆CaO₉), and Calcium lactate gluconate (C₉H₁₆CaO₁₀).

26. (canceled)

27. The method of claim 22, wherein gelling the solution comprises maintaining the solution at a temperature between about room temperature and about 60° C., preferably at about 37° C.

28. (canceled)

29. The method of claim 22, wherein calcining the dry gel comprises heating the dry gel to between about 400-600° C., or about 100-400° C.

30. The method of claim 29, wherein the heating comprises using a 3° C/min heating rate, followed by a 2 hour dwell, and then furnace cooling.

31-39. (canceled)
